(19) United States
(12) Patent Application Publication
(10) Pub. No.: US 2008/0208169 A1
(43) Pub. Date: Aug. 28, 2008

(54) Body-Space Treatment Catheter

(76) Inventors: Edward M. Boyle JR., Bend, OR (US); Nathan R. Every, Seattle, WA (US); Fred E. Silverstein, Seattle, WA (US); Steven Tallman, Everett, WA (US); Trevor J. Moody, Seattle, WA (US)

Correspondence Address: Paul J. Fordenbacher/Berkeley Law & Technology Gro;Berkeley Law & Technology Group LLP, 17933 NW Evergreen Parkway, Suite 250, Beaverton, OR, 97006, US

(21) Appl. No.: 10/559,983

(22) PCT Filed: Jun. 14, 2004
(86) PCT No.: PCT/US04/18749

§ 371 Date: May 1, 2008

Related U.S. Application Data
(60) Provisional application No. 60/477,689, filed on Jun. 11, 2003.

(51) Int. Cl. A61M 25/00

(52) U.S. Cl. 604/523

(57) ABSTRACT

A percutaneous treatment catheter comprises a canopy (12) having a treatment side adapted to provide at least one of a variety of treatments to a first tissue layer and a protection side that protects adjacent tissue from the treatment. Treatments include, but are not limited to, those that act to cause an inflammatory response resulting in forming scar tissue that would tend to form adhesions, such as, but not limited to, for the treatment of pleural effusions. In an embodiment, a treatment catheter comprises a shaft (20) having a shaft and a treatment head (10) disposed about the shaft distal end, the treatment head adapted to present a low profile in a closed state and a broad profile in a 10 deployed state, the treatment head adapted to percutaneously treat one of first and second tissue layers and protect the other of the first and second tissue layers from the treatment.

### RELATED APPLICATIONS

This application claims priority from U.S. Provisional Application Ser. No. 60/477,689, filed Jun. 11, 2003, the contents of which are hereby incorporated by reference as if recited in full herein.

### FIELD OF THE INVENTION

This invention generally relates to surgical tools and methods, and more particularly, tools and methods for treating body cavities.

### BACKGROUND OF THE INVENTION

Current methods for treating spaces within the body, such as those spaces defined between adjacent organs and between layers of adjacent body tissue layers, are somewhat ineffective and lead to patient discomfort. Examples of body spaces that may require therapy include: pleural space, pericardial space, peritoneal space, retroperitoneal space, wound spaces (hematoma, seroma), abscess cavities, joint spaces, reproductive organ spaces, genitourinary spaces, central nervous system spaces, airway spaces (upper and lower), among others.

For example, in certain lung diseases, the pleural space becomes enlarged due to fluid accumulation. Enlargement of the pleural space is detrimental for the patient, causing compression on the lungs and making breathing difficult. This is known as a pleural effusion. Pleural effusions are common in patients with end-stage heart disease, cancer, lung disease, or other medical problems. Pleural effusions are very disabling to the patient. Even small pleural effusions can cause symptoms such as shortness of breath and cough. When a pleural effusion is recognized clinically, it is imperative to establish a diagnosis and to try to treat the effusion so it goes away and does not come back.

The currently available treatments for patients with pleural effusions are frequently ineffective, painful, and require prolonged hospitalization. A common technique to treat a pleural effusion is to perform a pleurodesis. A pleurodesis is intended to induce a scar between the parietal and visceral pleura thereby fusing them together, to obliterate the pleural space and prevent the recurrence of pleural effusion. A pleurodesis procedure is generally palliative, and is performed based on the patients symptoms, underlying medical conditions, extent of disease, performance status and prognosis. A medical pleurodesis involves the chemical irritation of the pleural membranes. This can be done at the bedside, as an inpatient, with the instillation of a pleural irritant such as doxycycline or talc through a chest tube. These techniques require anywhere from 5 to 9 days hospitalization, with an average of one week in the hospital.

Unfortunately, even with the best techniques available today, pleurodesis fails in approximately one third of patients. Furthermore, the most promising sclerosing agent, talc, is loosing favor due to concern stemming from multiple reports of the induction of life threatening respiratory failure and systemic uptake (discussed below).

The enlarged pleural space can also be reduced or eliminated by inducing a scar one or both tissue layers of the pleural space. Often there are adjacent critical tissues and structures that need to be protected from the treatment, such as electrocautry, that is used to induce the tissue injury and subsequent scar. Such treatment requires invasive surgery to access the target tissue and to protect the untargeted tissue from damage, subjecting the patient to lengthy hospital stays and a protracted recovery period.

Which technique will replace talc pleurodesis is unknown. Thus, patients with symptomatic pleural effusions, and the physicians caring for them, are left with few options. Even the options currently available are not desirable, as the treatment is painful, it requires long hospitalizations, and the results are not consistently satisfying enough to justify the routine referral of patients with significant effusions for pleurodesis. Therefore, rather than putting a patient through a painful procedure with a long hospital stay, and an uncertain outcome, most clinicians caring for patients with end stage heart failure, cancer or other diseases with progressive effusions will simply have the patients tapped (thoracentesis) a few times, and usually the patients die within a month or two as the effusions come back.

Percutaneous and minimally invasive therapies are needed for the treatment of body space tissue that protects the surrounding non-targeted tissue. One reason why pleural effusion patients notoriously have recurrent pleural effusions, despite attempts at chemical pleurodesis, is that they are unable to mount an inflammatory response adequate enough to result in scarring between the pleural surfaces. This is because most are too sick to do so. Cancer patients are almost always malnourished. Many have been or are still on chemotherapy. Many have had radiation to the area. Some are on steroids for brain metastasis. The same can be said for end stage heart failure, cirrhotic, pneumonia patients and the like. Even when a chemical pleurodesis is attempted, the lack of inflammatory response can cause the procedure to fail in 30% or more of the patients. For this reason the most effective means to achieve a pleurodesis is to mechanically abrade, strip or burn the pleura, however, there are no tools to facilitate this via a thoracoscope, and utilizing a thoractomy to do this is often too morbid a procedure for end stage patients.

Talc has been used as a technique to induce an inflammatory response to induce apleurodesis. Talc either insufflated (poudrage) or in a suspension (slurry) was until recently commonly used to create a pleurodesis in patients with recurrent pneumothorax or recurrent pleural effusions. But there mounting evidence that talc is dangerous. There are now at least 52 cases in the literature in which patients developed the acute respiratory distress syndrome (ARDS) after receiving talc intrapleurally. Many of these patients went on to die. In one landmark study from Seattle by Rehse, Aye, and Florence, a review of patients undergoing talc pleurodesis was performed, documenting multiple respiratory and other complications. In their study, seventy-eight patients received 89 talc pleurodesis procedures. Respiratory complications or death occurred in 33%; and 9% of patients developed adult respiratory distress syndrome. (Am J Surg 1999 May; 177(5):437-40) The mechanism for the development of acute respiratory distress syndrome after the intrapleural administration of talc is not known, but it may be related to the systemic absorption of talc or contamination with endotoxin. (Current Opinion Pulmonary Med 2000 July; 6(4):255-8) Talc is a pulverized magnesium silicate preparation that varies from location to location, and distributor to distributor. Some hospitals make it up themselves, further reducing the usual quality assurances in the pharmaceutical industry. Ferrer and colleagues looked at the physical properties of eight talc preparations from distributors around the world. They found a wide range of particle sizes and varying degrees of impurities. (Chest 2001. 119: 1901-1905) This same group went on to study the significance of this in animals. They found that talc of varying particle size was demonstrated systemically in all the rabbits studied. (Chest. 2002, 122: 1018-27) Furthermore, they found that the smaller the particle size, the more effective the pleurodesis, however, the greater the systemic absorption. Other animal studies have shown talc is absorbed from the pleural space and is distributed to every organ in the body. (Chest 1999; 115 (1):190-3). To what degree this systemic absorption happens in humans is unknown, but it is indisputable that there have been a number of adverse effects from the use of talc and thus many are now refusing to use it. In fact, many hospitals will no longer allow the pharmacy to prepare talc due to the increasingly common reports of talc induced problems from systemic absorption.

The single most effective and reliable way to achieve a pleurodesis is by mechanically ablating the pleura at an open surgical operation. This ablative procedure is done by opening the chest or accessing it via multi port thoracoscopy and trying to strip or burn the pleura with electrocautry. Often times, at the end, talc is also administered. There are several issues that make the current surgical options less attractive. First, they are morbid, second, the often rely on the use of talc. Thoracoscopic pleurodesis with chest tube drainage is known as the most effective approach. The surgeon can add a partial pleurectomy where by the pleura is burned with the Bovie, or stripped from the chest wall to make sure there will be a scar formed. Surgically, a pleurectomy and pleural abrasion is generally effective in obliterating the pleural space and, thus, controlling the malignant pleural effusion. This procedure is done in many patients who undergo thoracotomy or thoracoscopy for an undiagnosed pleural effusion and are found to have malignancy. However, a total pleurectomy is a major surgical procedure associated with substantial morbidity and significant mortality. In fact, for malignant pleural effusions, a thoractomy has an operative mortality of nearly 10%. (Ann Thorac Surg 2002, 74:213-7) Furthermore, performing an adequate pleural ablation via thoracoscopy is technically difficult given the current tools.

Thus, current therapies for pleural effusions are either ineffective, morbid or too invasive. Thus there is a need for a more effective and less invasive form of therapy to obliterate this body space.

### SUMMARY OF THE INVENTION

In embodiments in accordance with the present invention, a percutaneous treatment catheter comprises a canopy having a treatment side adapted to provide at least one of a variety of treatments to a first tissue layer and a protection side that protects adjacent tissue from the treatment. Treatments include, but are not limited to, those that act to cause an inflammatory response resulting in forming scar tissue that would tend to form adhesions, such as, but not limited to, for the treatment of pleural effusions, and those treatments requiring a localized treatment, such as to treat a patch of cancer cells or tumor.

In embodiments of the present invention, one such class of treatments includes to cause an inflammatory response resulting in forming scar tissue that would tend to form adhesions, such as, but not limited to, for the treatment of pleural effusions. Such treatment includes ablation of the target tissue layer. Tissue ablation can be caused by the application of a suitable energy source delivered to the tissue, including electrocautery, cryogenic cooling, radio-frequency, harmonic vibration, laser energy, infrared, microwave, near infrared, ultrasound, photodynamic, direct heating, and chemical.

In an embodiment, a treatment catheter comprises a shaft having a shaft distal end and a shaft proximal end and a treatment head disposed about the shaft distal end, the treatment head adapted to present a low profile in a closed state and a broad profile in a deployed state, the treatment head adapted to percutaneously treat one of first and second tissue layers and protect the other of the first and second tissue layers from the treatment.

In another embodiment, the percutaneous treatment catheter comprises a treatment head with a canopy having a protection side facing a direction distal from the shaft and a treatment side facing a direction proximate the shaft. The canopy is supported by a frame assembly comprising a runner, a plurality of main ribs, a supporting rib coupled to each main rib, and an upper joint, the runner coupled to the shaft and moveable in an axial direction thereon. Each main rib has a main rib outer end and a main rib inner end pivotally coupled to the shaft distal end at the upper joint, each supporting rib having a supporting rib inner end pivotally coupled to the runner and a supporting rib outer end pivotally coupled to the main rib. The movement of the runner along the shaft from distal the upper joint to proximate the upper joint positions the frame assembly between a closed and deployed position, and therefore closes and deploys the canopy.

In another embodiment a treatment catheter wherein the treatment elements are resistive heating elements that provide a predetermined amount of heat. The treatment catheter of wherein the treatment elements are fiber optic elements that are adapted to provide a predetermined amount of laser energy. The treatment catheter wherein the treatment elements are adapted to discharge fluid. The treatment catheter wherein the treatment elements comprise radio-frequency emitting elements that provide a predetermined amount of RF.

In another embodiment a treatment catheter wherein the treatment head further comprising:

In another embodiment a canopy having a protection side facing a direction distal from the shaft and a treatment side facing a direction proximate the shaft, the canopy supported by a frame assembly comprising a runner, a plurality of main ribs, a supporting rib coupled to each main rib, and an upper joint, the runner coupled to the shaft and moveable in an axial direction thereon, each main rib having a main rib outer end and a main rib inner end pivotally coupled to the shaft distal end at the upper joint, each supporting rib having a supporting rib inner end pivotally coupled to the runner and a supporting rib outer end pivotally coupled to the main rib, wherein the movement of the runner along the shaft from distal the upper joint to proximate the upper joint positions the frame assembly between a closed and deployed position, and therefore closes and deploys the canopy.

In another embodiment a treatment catheter having a treatment head comprising an inflatable canopy having a protection side facing a direction distal from the shaft and a treatment side proximate the shaft, the inflatable canopy having a predefined shape such that when inflated, the treatment head takes the form of an umbrella, the shaft including an inner lumen adapted to supply a fluid to the canopy for inflation.

In another embodiment a treatment catheter having a treatment head comprising an inflatable canopy having a protection side facing a direction proximal to the shaft and a treatment side distal from the shaft, the inflatable canopy having a predefined shape such that when inflated, the treatment head takes the form of an umbrella, the shaft including an inner lumen adapted to supply a fluid to the canopy for inflation.

In another embodiment a treatment catheter having a treatment head comprising an inflatable treatment head disposed about the shaft distal end, the inflatable treatment head substantially axially bisected defining a protection side and a treatment side, the shaft including at least one inner lumen adapted to supply a fluid to the canopy for inflation.

In another embodiment, a treatment catheter having a treatment head comprising an inflatable treatment head disposed about the shaft distal end, the inflatable treatment head substantially axially bisected into a first balloon and a second balloon defining a protection balloon and a treatment balloon, the shaft including at least two inner lumens each adapted to supply a fluid to one of the protection balloon and a treatment balloon for inflation.

In an embodiment of a method of treating a first tissue layer while protecting a second tissue layer from treatment, comprising percutaneously placing a treatment catheter comprising, a shaft having a shaft distal end, a treatment head disposed about the shaft distal end, the treatment catheter adapted to present a low profile in a closed state and a broad profile in a deployed, the treatment catheter adapted to treat one of first and second tissue layers and protect the other of the first and second tissue layers from the treatment. Positioning a treatment side of a treatment head adjacent to the first tissue layer. Opening the treatment head and placing the treatment side in intimate contact with the first tissue layer. Treating the first tissue layer, closing the treatment head, and withdrawing the treatment catheter.

### DRAWINGS

FIG. 1 is a side cross-sectional view of a body section showing a body space characteristic of an effusion;

FIG. 2A is a side cross-sectional view of a pull-type treatment catheter deployed within the body section in accordance with an embodiment of the present invention;

FIG. 2B is a side cross-sectional view of the pull-type treatment catheter in a closed position;

FIG. 3 is a side cross-sectional view of a body section showing the body section upon removal of the treatment catheter and after tissue healing;

FIGS. 4A and 4B are side cross-sectional views of an embodiment of a pull-type treatment catheter, in the closed and deployed state, respectively, wherein the treatment side comprises treatment elements;

FIGS. 5A-5C are plan views of the treatment side showing various embodiments of arrangements of the treatment elements;

FIG. 6A is a side cross-sectional view of a push-type treatment catheter deployed within the body section, in accordance with an embodiment of the present invention;

FIG. 6B is a side cross-sectional view of the push-type treatment catheter in a closed position;

FIGS. 7A and 7B are side cross-sectional views of an embodiment of a push-type treatment catheter, in the closed and deployed state, respectively, wherein the treatment side comprises treatment elements;

FIGS. 8A and 8B are side cross-sectional views of an inflatable pull-type treatment catheter in a closed position and a deployed position, in accordance with an embodiment of the present invention;

FIGS. 9A and 9B are side cross-sectional views of an inflatable push-type treatment catheter in a closed position and a deployed position, in accordance with an embodiment of the present invention;

FIGS. 10A and 10B are side cross-sectional views of an inflatable treatment catheter in a deployed position and a closed position, in accordance with an embodiment of the present invention; and

FIG. 11 is a side cross-sectional view of a double-balloon inflatable treatment catheter in a deployed position, in accordance with an embodiment of the present invention.

### DESCRIPTION

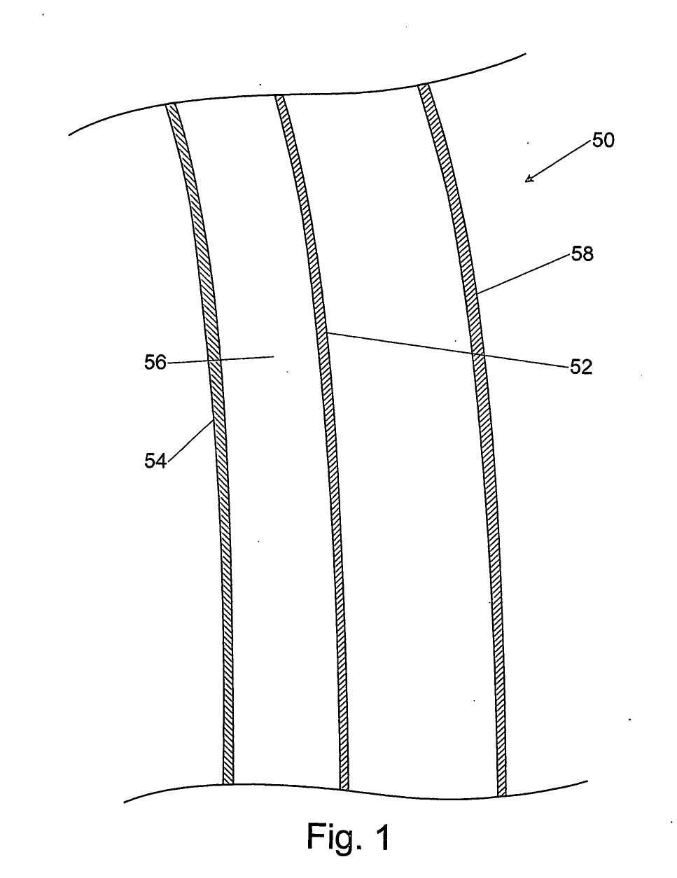

FIG. 1 is a side cross-sectional view of a body section 50 showing a body space 56 characteristic of an effusion. The body section 50 comprises a skin layer 58, a first tissue layer 52, a second tissue layer 54, and the body space 56 there between. Embodiments of the present invention provide methods and apparatus for treating one of the first and second tissue layers 52, 54 in order to close up the body space 56.

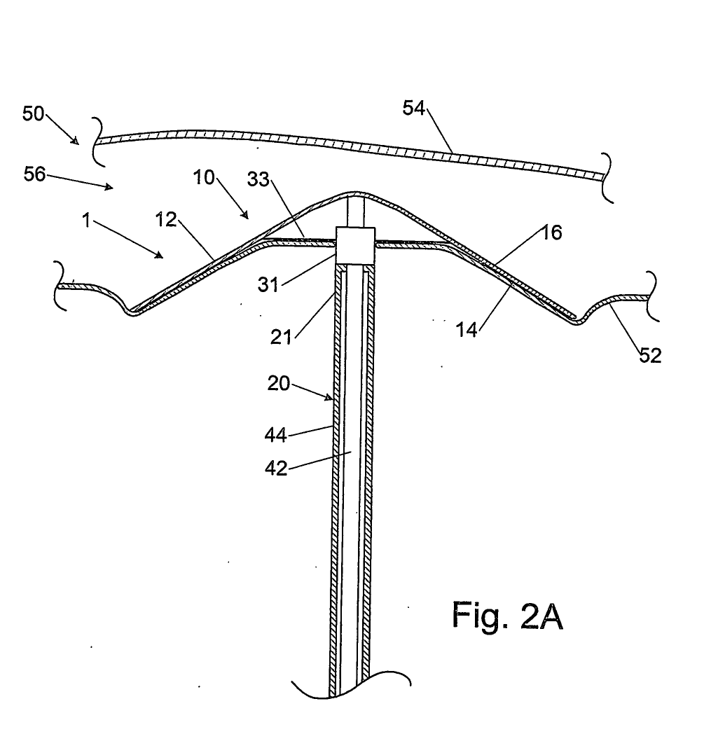

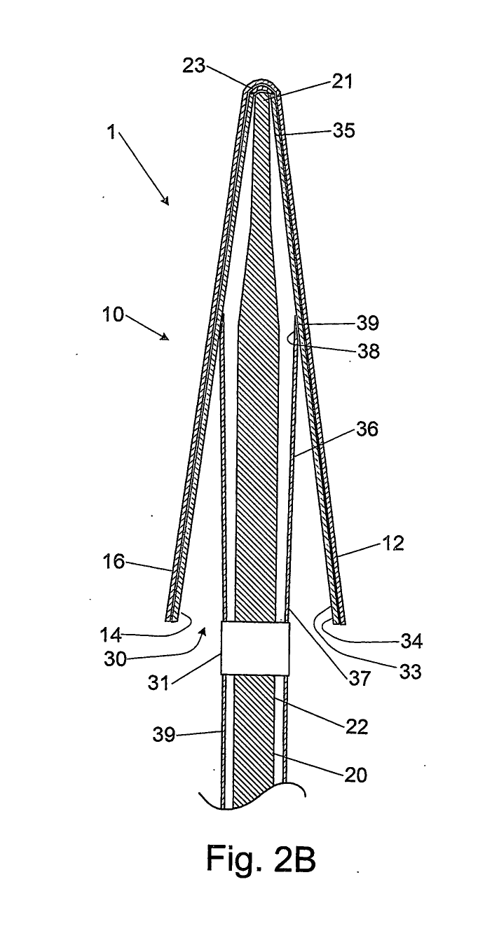

FIG. 2A is a side cross-sectional view of a treatment catheter 1 deployed within the body section 50 in accordance with an embodiment of the present invention. The treatment catheter 1 comprises a shaft 20 having a shaft distal end. An umbrella-shaped treatment head 10 is disposed about the shaft distal end 21. The treatment catheter 1 is adapted to present a low profile in a closed state and a broad profile in a deployed or open state. The treatment catheter 1 is adapted to treat one of the first and second tissue layers 52, 54 and protect the other of the first and second tissue layers 52, 54.

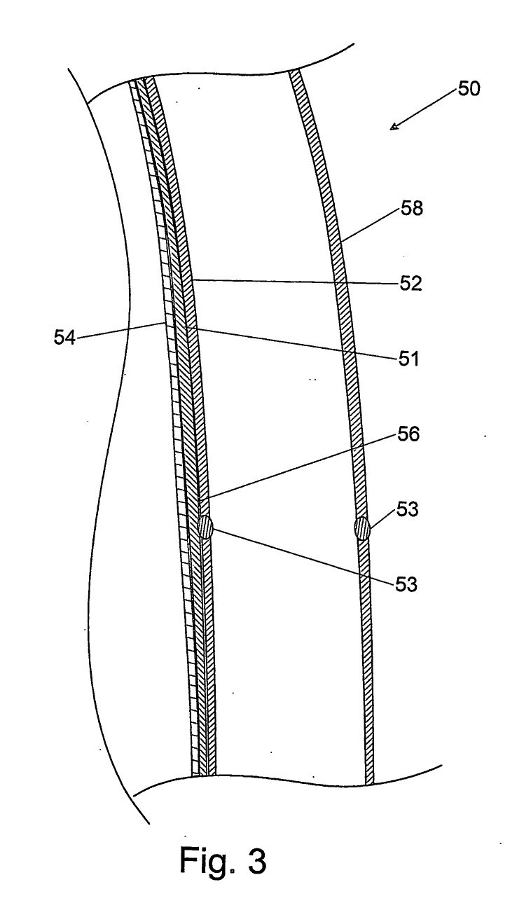

FIG. 3 is a side cross-sectional view of a body section showing the body section 50 upon removal of the treatment catheter 1 and after tissue healing. The body space 56 is closed being replaced by a scar layer 51 that act to adhere the first tissue layer 52 to the second tissue layer 54 eliminating the body space 56 there between. By way of using the treatment catheter 1 in this manner in one or more locations in the body space 56, the body space 56 can be effectively and permanently closed.

Referring again to FIG. 2A, shown is a side cross-sectional view of a pull-type treatment catheter 1 deployed within the body section 50, in accordance with an embodiment of the present invention. FIG. 2B is a side cross-sectional view of the pull-type treatment catheter 1 in a closed position. The pull-type treatment catheter 1 comprises a shaft 20 having a shaft distal end 21 and a shaft proximal end 22. Disposed about the shaft distal end 21 is a treatment head 10. The treatment head 10 comprises a canopy 12 having a protection side 16 facing a direction distal from the shaft 20 and a treatment side 14 proximate the shaft 20. The canopy 12 is supported by a frame assembly 30 comprising a runner 31, a plurality of main ribs 33, a supporting rib 36 coupled to each main rib 33, and an upper joint 23.

The runner 31 encircles the shaft 20 and is moveable in the axial direction thereon. Each main rib 33 has a main rib outer end 35 and a main rib inner end 34 pivotally coupled to the shaft distal end 21 at the upper joint 23. Each supporting rib 36 has a supporting rib inner end 37 that is pivotally coupled to the runner 31 and a supporting rib outer end 38 pivotally coupled to the main rib 33, such as, but not limited to about a location approximately half-way between the main rib inner end 34 and main rib outer end 35.

The movement of the runner 31 along the shaft 20 from distal the upper joint 23 to proximate the upper joint 23, positions the frame assembly 30 between a closed and deployed position, and therefore closes and deploys the canopy 12. The movement of the runner 31 is activated by advancing and withdrawing a runner actuator 44, as shown in FIG. 2A.

The pull-type treatment catheter 1 is adapted for percutaneous placement of the treatment head 10 within the body space 56 and deployed, as shown in FIG. 2A. After the canopy 12 is opened, the treatment side 14 is placed adjacent the first tissue layer 52 as well as placing the protection side 16 adjacent the second tissue layer 54. A pulling motion by the operator on the shaft 20 effectively places a portion of the first tissue layer 52 in intimate contact with the treatment side 14 of the canopy 12 and separates the first tissue layer 52 from the second tissue layer 54. Treatment of the first tissue layer 52 can now take place without affecting the second tissue layer 52.

Percutaneous placement of the treatment head 10 within the body space 56 is performed by any known technique suitable for the particular purpose. Suitable techniques for placing catheters, such as angioplasty catheters, are generally known in the art. Techniques known as over-the-wire involve the placement of a wire, or in some cases a needle, to the treatment site, and advancing the treatment catheter over the wire which acts as a guide to properly place the treatment head 10. Embodiments of the present invention include a central lumen (shown in FIG. 2A, for example) that runs axially through the shaft 10 to allow over-the-wire placement.

Another technique generally known in the art is known to include placement of a tube to the desired treatment site and passing the treatment catheter 1 through the lumen of the tube, then withdrawing the tube. The tube acts as a guide to properly place the treatment head 10. Embodiments of the present invention that include a central lumen (shown in FIG. 2A, for example) that runs axially through the shaft 10 or a solid shaft can be placed with this technique.

Visualization of the treatment catheter is provided by methods known in the art. Such methods include, but not limited to, the use of an endoscope to directly visualize the treatment catheter 1. Another method includes, but not limited to, radiological guidance, wherein a radiopaque marker is used strategically on the treatment catheter for visualization with x-ray or other radiation.

Axial stiffness of the treatment catheter 1 is predetermined suitable for a particular purpose. The pull-type treatment catheter 1 can have less axial stiffness if it is guided into position by a tube, as it will not be required to pass-through tissue and the like. The axial stiffness of the treatment catheter 1 will require a higher axial stiffness for over-the-wire or direct placement techniques.

In embodiments in accordance with the present invention, the treatment side 14 of the canopy 12 is adapted to provide at least one of a variety of treatments to the first tissue layer 52. Treatments include, but are not limited to, those that act to cause an inflammatory response resulting in forming scar tissue that would tend to form adhesions, such as, but not limited to, for the treatment of pleural effusions, and those treatments requiring a localized treatment, such as to treat a patch of cancer cells or tumor.

In embodiments of the present invention, one such class of treatments includes to cause an inflammatory response resulting in forming scar tissue that would tend to form adhesions, such as, but not limited to, for the treatment of pleural effusions. Such treatment includes ablation of the target tissue layer. Tissue ablation can be caused by the application of a suitable energy source delivered to the tissue, including electrocautery, cryogenic cooling, radio-frequency, harmonic vibration, laser energy, infrared, microwave, near infrared, ultrasound, photodynamic, direct heating, and chemical.

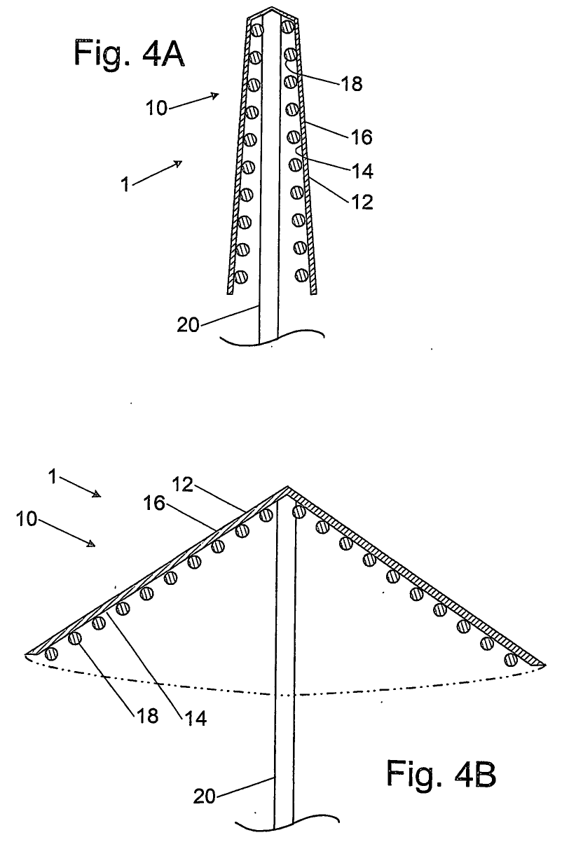

Referring again to FIG. 2A, the pull-type treatment catheter 1 can be effectively used to place the first tissue layer 52 in intimate contact with the treatment side 14 of the canopy 12. FIGS. 4A and 4B are side cross-sectional views of an embodiment of a pull-type treatment catheter 1, in the closed and deployed state, respectively, wherein the treatment side 14 comprises treatment elements 18. Treatment elements 18 can be any number of devices, such as one or more current conductive elements, such as electric wire. The treatment elements 18 are effectively isolated from the second tissue layer 54 by the protection side 16 of the canopy 12 and by the distance between the first tissue layer 23 and the second tissue layer 54 afforded by pulling the shaft 20 of the treatment catheter 1.

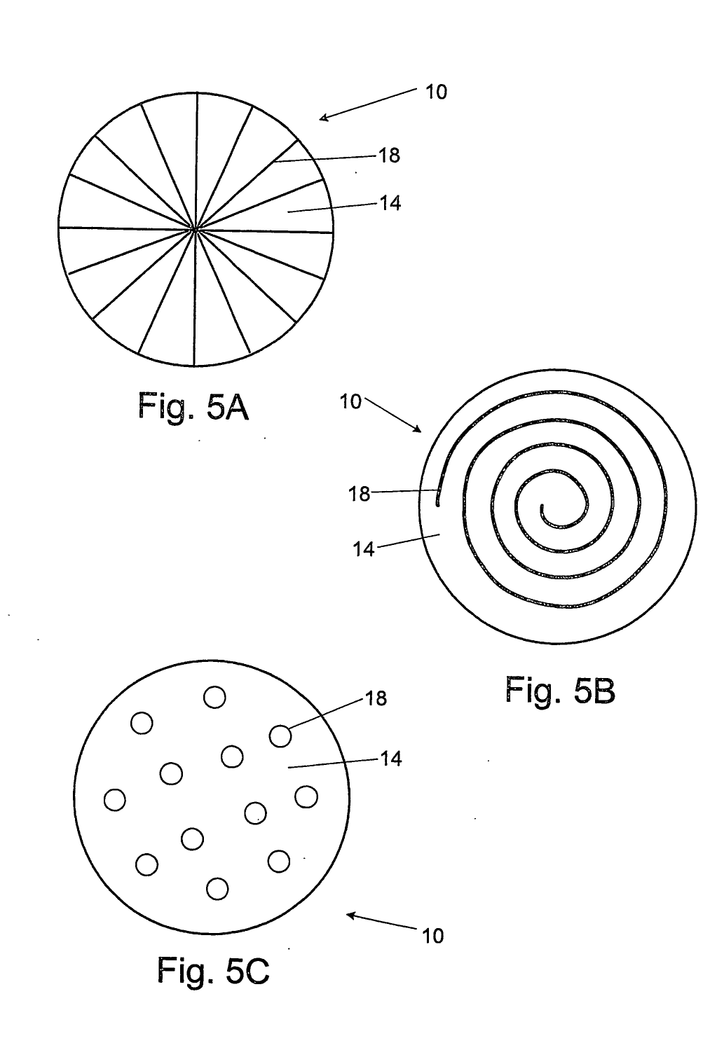

FIGS. 5A-5C are plan views of the treatment side 14 showing various embodiments of arrangements of the treatment elements 18, among others. FIG. 5A illustrates treatment elements 18 that radiate from a central portion of the treatment side 14, suitable for a particular purpose. FIG. 5B illustrates treatment elements 18 that radiate in a spiral pattern from a central portion of the treatment side 14, suitable for a particular purpose. FIG. 5C illustrates treatment elements 18 that present in discrete locations on the treatment side 14, suitable for a particular purpose.

In an embodiment in accordance with the present invention, the treatment elements 18 are resistive heating elements that provide a predetermined amount of heat to the first tissue layer 23, suitable for a particular purpose.

In another embodiment in accordance with the present invention, the treatment elements 18 are fiber optic elements that provide a predetermined amount of laser energy to the first tissue layer 23, suitable for a particular purpose.

In another embodiment in accordance with the present invention, the treatment elements 18 are fluid-carrying elements that provide a predetermined amount of heat or cryogenic cooling to the first tissue layer 23, suitable for a particular purpose.

In another embodiment in accordance with the present invention, the treatment elements 18 are radio-frequency (RF) emitting elements that provide a predetermined amount of RF to the first tissue layer 23, suitable for a particular purpose.

In another embodiment in accordance with the present invention, the treatment elements 18 are fluid-eluding elements that provide a predetermined amount of treatment fluid to the first tissue layer 23. Such treatment fluid includes, but is not limited to, pharmaceutical compounds and inflammation-producing compounds, suitable for a particular purpose.

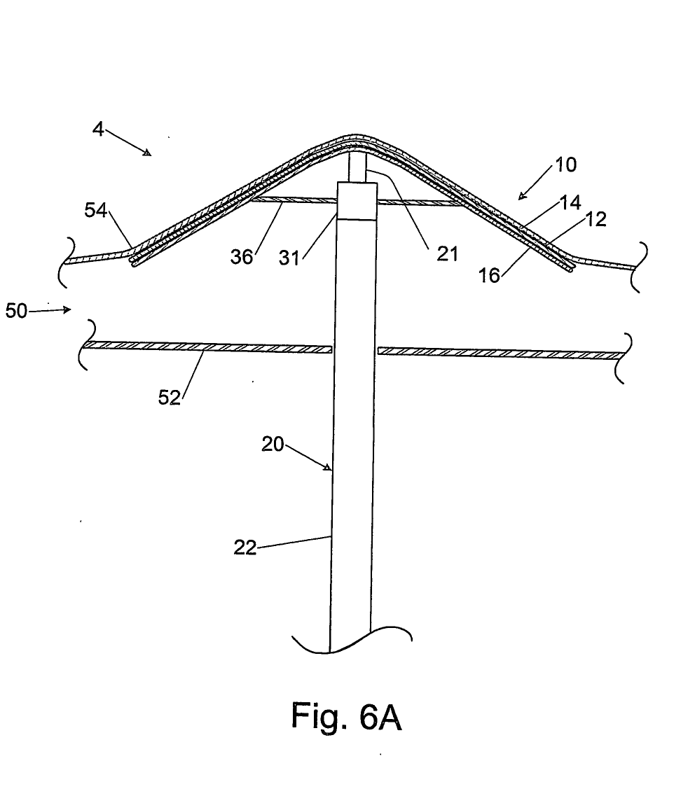

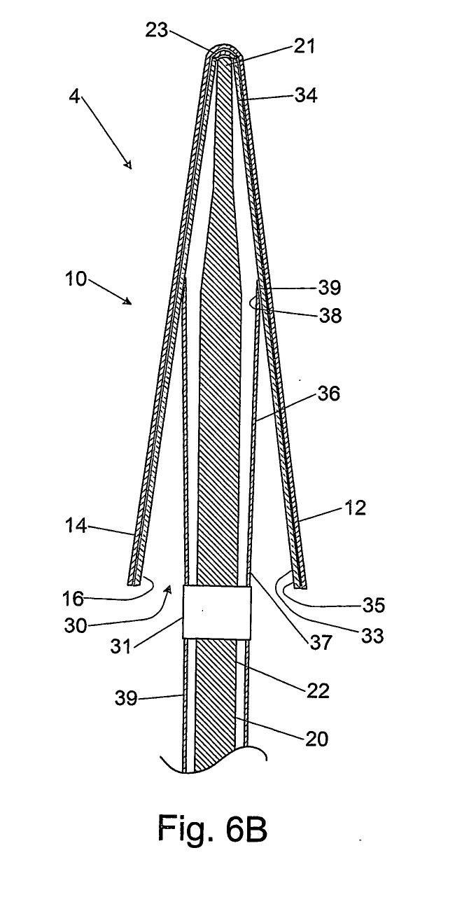

FIG. 6A is a side cross-sectional view of a push-type treatment catheter 4 deployed within the body section 50, in accordance with an embodiment of the present invention. FIG. 6B is a side cross-sectional view of the push-type treatment catheter 4 in a closed position. The push-type treatment catheter 4 comprises a shaft 20 having a shaft distal end 21 and a shaft proximal end 22. Disposed about the shaft distal end 21 is a treatment head 10. The treatment head 10 comprises a canopy 12 having a protection side 16 facing a direction proximal to the shaft 20 and a treatment side 14 distal from the shaft 20. The canopy 12 is supported by a frame assembly 30 of substantially the same configuration as presented above for FIG. 2A.

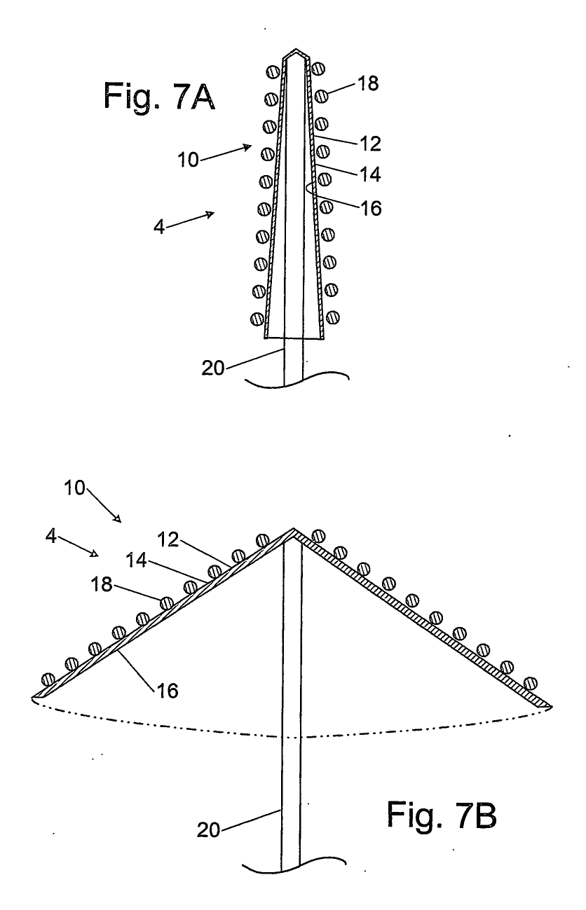

The push-type treatment catheter 4 can be effectively used to place the second tissue layer 54 in intimate contact with the treatment side 14 of the canopy 12. FIGS. 7A and 7B are side cross-sectional views of an embodiment of a push-type treatment catheter 4, in the closed and deployed state, respectively, wherein the treatment side 14 comprises treatment elements 18. The treatment elements 18 are as substantially described for FIGS. 4A, 4B and 5A-5C, above.

The push-type treatment catheter 4 is adapted for percutaneous placement of the treatment head 10 within the body space 56 and deployed, as shown in FIG. 6A. After the canopy 12 is opened, the treatment side 14 is placed adjacent the second tissue layer 54 as well as placing the protection side 16 adjacent the first tissue layer 53. A pushing motion by the operator on the shaft 20 effectively places a portion of the second tissue layer 54 in intimate contact with the treatment side 14 of the canopy 12 and separates the first tissue layer 52 from the second tissue layer 54. Treatment of the second tissue layer 54 can now take place without affecting the first tissue layer 54.

Axial stiffness of the push-type treatment catheter 4 is predetermined suitable for a particular purpose. The push-type treatment catheter 4 requires a relatively higher axial stiffness compared with the pull-type treatment catheter 1, as sufficient stiffness is required to push against the tissue during treatment.

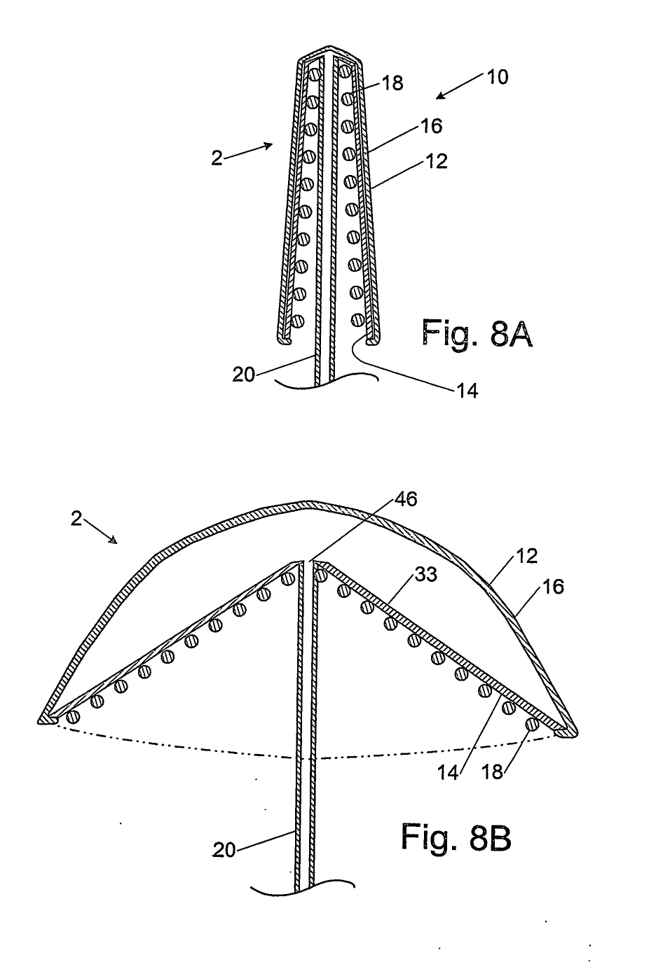

FIGS. 8A and 8B are side cross-sectional views of an inflatable pull-type treatment catheter 2 in a closed position and a deployed position, in accordance with an embodiment of the present invention. The pull-type treatment catheter 2 comprises a shaft 20 having a shaft distal end 21 and a shaft proximal end 22. Disposed about the shaft distal end 21 is a treatment head 10. The treatment head 10 comprises an inflatable canopy 12 having a protection side 16 facing a direction distal from the shaft 20 and a treatment side 14 proximate the shaft 20.

The inflatable canopy 12 has a predefined shape such that when inflated, the treatment head takes the form of an umbrella.

In another embodiment, the canopy 12 is supported by a frame assembly 30 comprising a runner 31, a plurality of main ribs 33, a supporting rib 36 coupled to each main rib 33, and an upper joint 23, substantially as shown in FIG. 2A. The movement of the runner 31 along the shaft 20 from distal the upper joint 23 to proximate the upper joint 23, positions the frame assembly 30 between a closed and deployed position, and therefore closes and deploys the canopy 12. The inflatable canopy providing a feature to support the second tissue layer 54 farther from the treatment side 14.

As in the other embodiments, the inflatable pull-type treatment catheter 4 is adapted for percutaneous placement of the treatment head 10 within the body space 56 and deployed. After the canopy 12 is opened, the treatment side 14 is placed adjacent the first tissue layer 52 as well as placing the protection side 16 adjacent the second tissue layer 54. A pulling motion by the operator on the shaft 20 effectively places a portion of the first tissue layer 52 in intimate contact with the treatment side 14 of the canopy 12 and separates the first tissue layer 52 from the second tissue layer 54. Treatment of the first tissue layer 52 can now take place without affecting the second tissue layer 52.

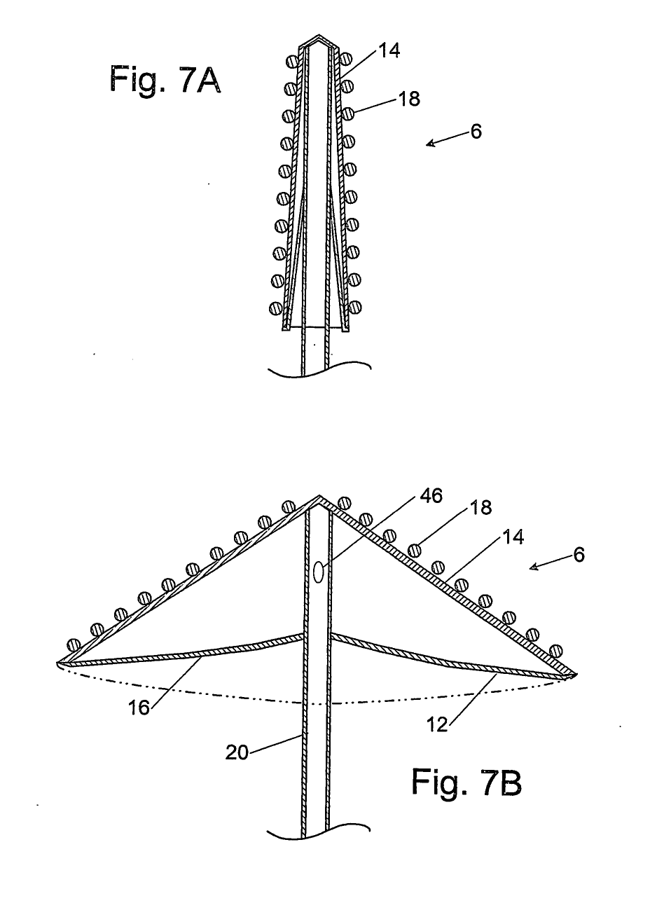

FIGS. 9A and 9B are side cross-sectional views of an inflatable push-type treatment catheter 5 in a closed position and a deployed position, in accordance with an embodiment of the present invention. The inflatable push-type treatment catheter 5 comprises a shaft 20 having a shaft distal end 21 and a shaft proximal end 22. Disposed about the shaft distal end 21 is a treatment head 10. The treatment head 10 comprises an inflatable canopy 12 having a protection side 16 facing a direction proximal to the shaft 20 and a treatment side 14 distal from the shaft 20.

The inflatable canopy 12 has a predefined shape such that when inflated, the treatment head takes the form of an umbrella. The shaft has an inner lumen (not shown) to supply a fluid to the canopy for inflation. Method for inflating distal balloons, such as angioplasty catheters is well known in the art and suitable for use herewith.

In another embodiment, the canopy 12 is supported by a frame assembly 30 comprising a runner 31, a plurality of main ribs 33, a supporting rib 36 coupled to each main rib 33, and an upper joint 23, substantially as shown in FIG. 2A. The movement of the runner 31 along the shaft 20 from distal the upper joint 23 to proximate the upper joint 23, positions the frame assembly 30 between a closed and deployed position, and therefore closes and deploys the canopy 12. The inflatable canopy providing a feature to support the second tissue layer 54 farther from the treatment side 14.

As in the other embodiments, the inflatable push-type treatment catheter 5 is adapted for percutaneous placement of the treatment head 10 within the body space 56 and deployed. After the canopy 12 is opened, the treatment side 14 is placed adjacent the second tissue layer 54 as well as placing the protection side 14 adjacent the first tissue layer 52. A pushing motion by the operator on the shaft 20 effectively places a portion of the second tissue layer 54 in intimate contact with the treatment side 14 of the canopy 12 and separates the first tissue layer 52 from the second tissue layer 54. Treatment of the second tissue layer 54 can now take place without affecting the first tissue layer 54.

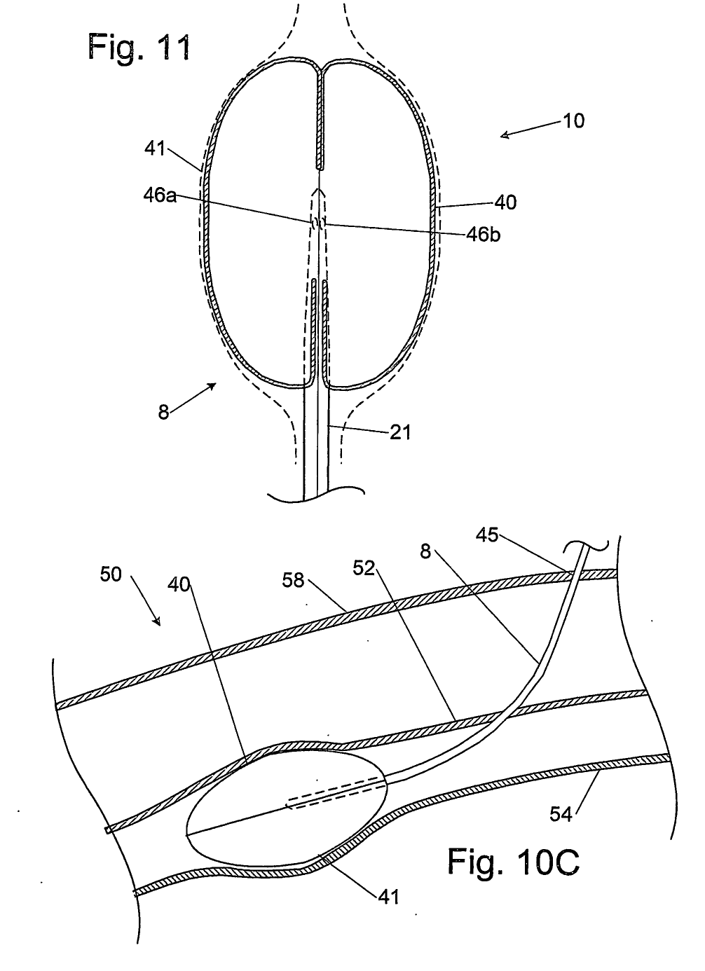

FIGS. 10A and 10B are side cross-sectional views of an inflatable treatment catheter 7 in a deployed position and a closed position, in accordance with an embodiment of the present invention. The inflatable treatment catheter 7 comprises a shaft 20 having a shaft distal end 21 and a shaft proximal end 22. Disposed about the shaft distal end 21 is an inflatable treatment head 10. The inflatable treatment head 10 is substantially axially bisected defining a protection side 16 and a treatment side 14.

The inflatable treatment catheter 6 is adapted for percutaneous placement of the inflatable treatment head 10 within the body space 56. FIG. 10C is a side cross-sectional view of the inflatable treatment catheter 6 with the inflatable treatment head 10 inflated and deployed within the body space 56. After the inflatable treatment head 10 is inflated, the treatment side 14 is positioned adjacent to and in intimate-contact with the first tissue layer 52 as well as the protection side 16 is positioned adjacent the second tissue layer 54 effectively separating the first and second tissue layers 52, 54.

A pulling motion by the operator on the shaft 20 moves the inflatable treatment head 10 within the body space 56 and effectively places a portion of the first tissue layer 52 in intimate contact with the treatment side 14 of the canopy 12 and separates the first tissue layer 52 from the second tissue layer 54. Treatment of the first tissue layer 52 can now take place without affecting the second tissue layer 52 at each location of the inflatable treatment head 10.

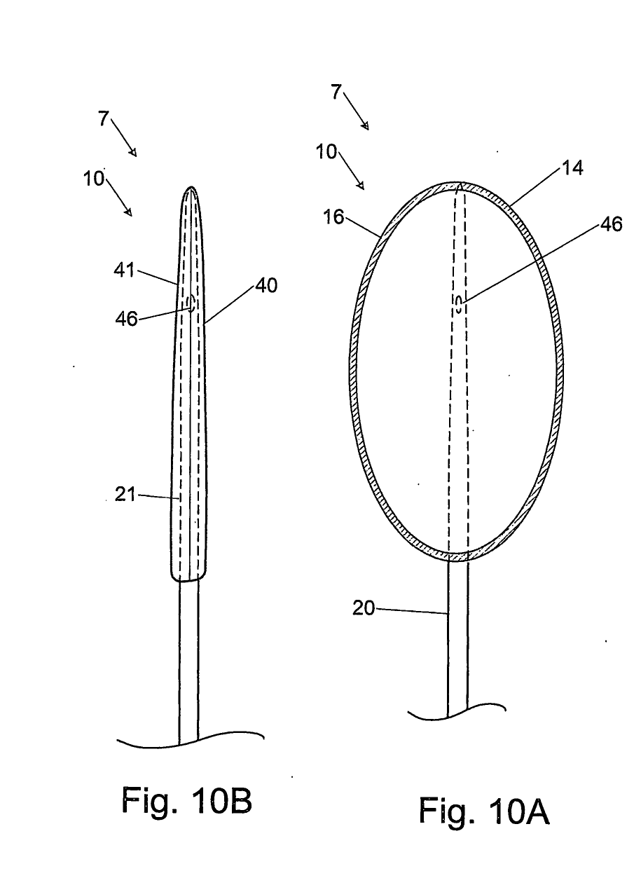

FIG. 11 is a side cross-sectional view of a double-balloon inflatable treatment catheter 8 in a deployed position, in accordance with an embodiment of the present invention. The double-balloon inflatable treatment catheter 8 comprises a shaft 20 having a shaft distal end 21 and a shaft proximal end 22. Disposed about the shaft distal end 21 is a double-balloon inflatable treatment head 10. The double-balloon inflatable treatment head 10 comprises a treatment balloon 40 and a protection balloon 41, the intersection of which is approximately axially bisecting the double-balloon treatment head 10 defining a protection side 16 and a treatment side 14. The individual treatment balloon 40 and a protection balloon 41 allows for additional capability for treatment options. In an embodiment in accordance with the present invention, each of the treatment balloon 40 and a protection balloon 41 are inflated at a different pressure to accommodate various anatomical features.

In another embodiment in accordance with the present invention, each of the treatment balloon 40 and a protection balloon 41 are inflated with different fluids, for example, a treatment fluid that is discharged from the treatment side 14 and an inflation fluid that does not discharge from the protection side 16.

Embodiments of methods for using the treatment devices provided above include a variety of medical procedures, some of which are provided herein, among others.

Thermal ablation for the treatment of pleurodysis. In this embodiment, heat is used to irritate the pleural surfaces on one or both tissue surfaces, the visceral and parietal pleura, for example, to cause granulation formation, adhesion, fibrosis and closure of the body space.

Laser ablation for the treatment of pleurodysis. In this embodiment, laser is used at one of a variety of frequencies. Nd:YAG or Argon laser energy can be directed to the treatment surface via a flexible wave guide. CO2 laser energy typically needs a whispering wave guide or an open channel for transmission. Other lasers of single or multiple (two or more) wave lengths, include dual photon lasers, among others. The laser is directed towards one or both tissue surfaces, the visceral pleura (VP) and parietal pleura (PP), to heat and abrade the lining of the pleural space. This can occur in a random pattern or in a pattern that insures that the treated areas on either surface PP or VP will be aligned and will touch each other when the pleural space is emptied of fluid (liquid and/or gas). This pattern may be critical to achieve closure without having to heat excessively large areas of pleura.

The treatment pattern made with a laser, perhaps with a robotic controller, matches the two layers (PP and VP) so that when fluid (liquid and/or gas) is removed, the PP and VP tissues touch and undergo fibrosis to close the pleural space.

Electrocautery for the treatment of pleurodysis. Electrocautery can be used in several ways to heat, and/or abrade the PP and VP. This includes monopolar, bipolar, multipolar, electrofulguration, and spark gap gas assisted types of techniques (Beacon technology). In some instances, a ground plate is needed and in others it is not.

Microwave energy for the treatment of pleurodysis. This embodiment uses external antennae or internal antennae to direct microwave energy to heat the treatment tissue layer and cause a pleurodysis.

Infrared energy for the treatment of pleurodysis. Infrared energy can be used to irritate the VP and PP to cause pleurodysis.

Near infrared energy for the treatment of pleurodysis. Near infrared energy produces heating to induce pleurodysis.

Ultrasound energy for the treatment of pleurodysis. Ultrasound energy or high frequency focused ultrasound (HIFU) is used to cause abrasion of tissue through heating. This energy can be directed either from inside the pleural space or outside the space to heat tissue by energy absorption and cavitation to cause pleurodysis. In most instances a fluid or other guidepath is needed to get the ultrasound to the tissue target. Embodiments of the inflatable treatment device can be inflated with a liquid that can transmit ultrasound energy to the treatment tissue.

The treatment side 14 can be made thinner and the protection side 16 can be made thicker to preferentially transmit the ultrasound energy to the treatment side 14 while protecting the protection side 16.

Photodynamic dye with heating for the treatment of pleurodysis. Photodynamic dye injected systemically can be heated using an appropriate wavelength of light to cause pleurodysis.

Direct heating for the treatment of pleurodysis. The treatment side 14 is heated to apply heat directly to the VP or PP to cause pleurodysis. Heating can be rapid or slow and can use a variety of mechanisms in the treatment device to heat (electrical, chemical, laser, etc.).

Chemical irritation for the treatment of pleurodysis. A chemical can be discharged from the treatment side and directed against the PP or VP to irritate the surfaces to cause pleurodysis. This can be direct irritation or chemical heating of the surface. A chemical or placement of microparticles or microspheres can be used to irritate the VP or PP chemically. A chemical substance either naturally occurring, such as, but not limited to, animal or human collagen or fibrin, or synthetic can be used to adhere to the VP and PP and to close the pleural space. This action can be slow or rapid. It can be isothermal or thermal. Polymer liquid directed against the PP and VS. This can induce immediate adhesion or require activation to be adherent. Once activated, the adhesive sticks to the VP and PP and to itself and closes the pleural space.

Mechanical ablation for the treatment of pleurodysis. The treatment side 14 is adapted to present an abrasive surface for mechanical abrading against either the VP or PP or moved against the VP or PP. Mechanical abrasion causes a lesion leading to pleurodysis.

Microspheres for the treatment of pleurodysis. Material comprising microspheres are placed into the pleural space which conform to the body space to be closed. Once in position and with fluid (liquid and/or gas) evacuated, the material is activated to adhere to the VP and PP and close the pleural space.

Chips or other physical forms of polymer are introduced into the pleural space to be activated in the same way as the microspheres. The chips contain the adherent material as well as the activator in a pattern such that when activated, the activator encounters the adherent material and causes the material to go from the non-adherent form to the adherent form to close the pleural space.

Embodiments in accordance with the present invention deposit a material into the body space. Such material can be naturally occurring, such as, but not limited to, animal or human collagen or fibrin, or synthetic, such as, but not limited to, polymer. The material should have one or more of the following characteristics:
1. That the material be adherent to the pleura on both sides PP and VP
2. That the material be somewhat flexible to allow movement of the chest wall
3. That the material be biocompatible and last for months to years
4. That the material be in one form (solid or liquid) and then take another shape with a stimulus modification such as heat, electric current, light, chemical interaction, etc.
5. That the material be liquid so that it can be painted onto the target surface, or sprayed on, or solid so that it can be formed into a wafer, balloon, net, disk, etc.
6. To contain a material which is radiographically visible or acoustically visible so that the position can be confirmed after placement. The material may need to be seen endoscopically as well.

Although specific embodiments have been illustrated and described herein for purposes of description of the preferred embodiment, it will be appreciated by those of ordinary skill in the art that a wide variety of alternate and/or equivalent implementations calculated to achieve the same purposes may be substituted for the specific embodiment shown and described without departing from the scope of the present invention. Those with skill in the art will readily appreciate that the present invention may be implemented in a very wide variety of embodiments. This application is intended to cover any adaptations or variations of the embodiments discussed herein. Therefore, it is manifestly intended that this invention be limited only by the claims and the equivalents thereof.

We claim:

1. A treatment catheter comprising:
a shaft having a shaft distal end and a shaft proximal end;
a treatment head disposed about the shaft distal end, the treatment head adapted to present a low profile in a closed state and a broad profile in a deployed state, the treatment head adapted to percutaneously treat one of first and second tissue layers and protect the other of the first and second tissue layers from the treatment.

2. The treatment catheter of claim 1, the treatment head further comprising:
a canopy having a protection side facing a direction distal from the shaft and a treatment side facing a direction proximate the shaft, the canopy supported by a frame assembly comprising a runner, a plurality of main ribs, a supporting rib coupled to each main rib, and an upper joint, the runner coupled to the shaft and moveable in an axial direction thereon, each main rib having a main rib outer end and a main rib inner end pivotally coupled to the shaft distal end at the upper joint, each supporting rib having a supporting rib inner end pivotally coupled to the runner and a supporting rib outer end pivotally coupled to the main rib, wherein the movement of the runner along the shaft from distal the upper joint to proximate the upper joint positions the frame assembly between a closed and deployed position, and therefore closes and deploys the canopy.

3. The treatment catheter of claim 2, wherein the treatment side comprises treatment elements.

4. The treatment catheter of claim 3, wherein the treatment elements radiate from a central portion of the treatment side.

5. The treatment catheter of claim 3, wherein the treatment elements radiate in a spiral pattern from a central portion of the treatment side.

6. The treatment catheter of claim 3, wherein the treatment elements are present in discrete locations on the treatment side.

7. The treatment catheter of claim 3, wherein the treatment elements are resistive heating elements that provide a predetermined amount of heat.

8. The treatment catheter of claim 3, wherein the treatment elements are fiber optic elements that are adapted to provide a predetermined amount of laser energy.

9. The treatment catheter of claim 3, wherein the treatment elements are adapted to discharge fluid.

10. The treatment catheter of claim 3, wherein the treatment elements comprise radio-frequency emitting elements that provide a predetermined amount of RF.

11. The treatment catheter of claim 1, the treatment head further comprising:
a canopy having a protection side facing a direction distal from the shaft and a treatment side facing a direction proximate the shaft, the canopy supported by a frame assembly comprising a runner, a plurality of main ribs, a supporting rib coupled to each main rib, and an upper joint, the runner coupled to the shaft and moveable in an axial direction thereon, each main rib having a main rib outer end and a main rib inner end pivotally coupled to the shaft distal end at the upper joint, each supporting rib having a supporting rib inner end pivotally coupled to the runner and a supporting rib outer end pivotally coupled to the main rib, wherein the movement of the runner along the shaft from distal the upper joint to proximate the upper joint positions the frame assembly between a closed and deployed position, and therefore closes and deploys the canopy.

12. The treatment catheter of claim 1, the treatment head further comprising:
an inflatable canopy having a protection side facing a direction distal from the shaft and a treatment side proximate the shaft, the inflatable canopy having a predefined shape such that when inflated, the treatment head takes the form of an umbrella, the shaft including an inner lumen adapted to supply a fluid to the canopy for inflation.

13. The treatment catheter of claim 12, the treatment head further comprising:
a frame assembly supporting the canopy, the frame assembly comprising a runner, a plurality of main ribs, a supporting rib coupled to each main rib, and an upper joint, the runner coupled to the shaft and moveable in an axial direction thereon, each main rib having a main rib outer end and a main rib inner end pivotally coupled to the shaft distal end at the upper joint, each supporting rib having a supporting rib inner end pivotally coupled to the runner and a supporting rib outer end pivotally coupled to the main rib, wherein the movement of the runner along the shaft from distal the upper joint to proximate the upper joint positions the frame assembly between a closed and deployed position, and therefore closes and deploys the canopy.

14. The treatment catheter of claim 1, the treatment head further comprising:
an inflatable canopy having a protection side facing a direction proximal to the shaft and a treatment side distal from the shaft, the inflatable canopy having a predefined shape such that when inflated, the treatment head takes the form of an umbrella, the shaft including an inner lumen adapted to supply a fluid to the canopy for inflation.

15. The treatment catheter of claim 14, the treatment head further comprising:
a frame assembly supporting the canopy, the frame assembly comprising a runner, a plurality of main ribs, a supporting rib coupled to each main rib, and an upper joint, the runner coupled to the shaft and moveable in an axial direction thereon, each main rib having a main rib outer end and a main rib inner end pivotally coupled to the shaft distal end at the upper joint, each supporting rib having a supporting rib inner end pivotally coupled to the runner and a supporting rib outer end pivotally coupled to the main rib, wherein the movement of the runner along the shaft from distal the upper joint to proximate the upper joint positions the frame assembly between a closed and deployed position, and therefore closes and deploys the canopy.

16. The treatment catheter of claim 1, the treatment head further comprising:
an inflatable treatment head disposed about the shaft distal end, the inflatable treatment head substantially axially bisected defining a protection side and a treatment side, the shaft including at least one inner lumen adapted to supply a fluid to the canopy for inflation.

17. The treatment catheter of claim 1, the treatment head further comprising:
an inflatable treatment head disposed about the shaft distal end, the inflatable treatment head substantially axially bisected into a first balloon and a second balloon defining a protection balloon and a treatment balloon, the shaft including at least two inner lumens each adapted to supply a fluid to one of the protection balloon and a treatment balloon for inflation.

18. The treatment catheter of claim 1, the shaft further comprising a first lumen configured and dimensioned to receive a guide wire for directing the catheter.

19. Method of treating a first tissue layer while protecting a second tissue layer from treatment, comprising:
percutaneously placing a treatment catheter comprising:
a shaft having a shaft distal end;
a treatment head disposed about the shaft distal end, the treatment catheter adapted to present a low profile in a closed state and a broad profile in a deployed, the treatment catheter adapted to treat one of first and second tissue layers and protect the other of the first and second tissue layers from the treatment;
positioning a treatment side of a treatment head adjacent to the first tissue layer,
opening the treatment head;
placing the treatment side in intimate contact with the first tissue layer;
treating the first tissue layer,
closing the treatment head; and
withdrawing the treatment catheter.

20. The method of claim 19, wherein placing the treatment side in intimate contact with the first tissue layer comprises:
pulling on the treatment catheter to place the treatment side in intimate contact with the first tissue layer.

21. The method of claim 19, wherein placing the treatment side in intimate contact with the first tissue layer comprises:
pushing on the treatment catheter to place the treatment side in intimate contact with the first tissue layer.

22. The method of claim 19, wherein treating the first tissue layer comprises:
ablating the first tissue layer.

23. The method of claim 22, wherein ablating the first tissue layer comprises:
electrocauterising the first tissue layer.

24. The method of claim 22, wherein ablating the first tissue layer comprises:
cryogenically cooling the first tissue layer.

25. The method of claim 22, wherein ablating the first tissue layer comprises:
ablating the first tissue layer using radio-frequency energy.

26. The method of claim 22, wherein ablating the first tissue layer comprises:
ablating the first tissue layer using harmonic vibration.

27. The method of claim 22, wherein ablating the first tissue layer comprises:
ablating the first tissue layer using laser energy.

28. The method of claim 22, wherein ablating the first tissue layer comprises:
ablating the first tissue layer using infrared energy.

29. The method of claim 22, wherein ablating the first tissue layer comprises:
ablating the first tissue layer using ultrasound energy.

30. The method of claim 22, wherein ablating the first tissue layer comprises:
ablating the first tissue layer using chemical stimulation.

31. The method of claim 19, wherein treating the first tissue layer comprises:
applying medicine for an effective period of time to treat a body space condition.

32. The method of claim 19, wherein treating the first tissue layer comprises:
applying a chemical pleurodesant agent for an effective period of time to treat a body space condition.

33. The method of claim 19, wherein treating the first tissue layer comprises:
applying a chemotherapeutic drugs for an effective period of time to treat a body space condition.

34. The method of claim 19, wherein treating the first tissue layer comprises:
applying an antibiotics for an effective period of time to treat a body space condition.
